(19) United States
(12) Patent Application Publication
(10) Pub. No.: US 2015/0328269 A1
(43) Pub. Date: Nov. 19, 2015

(54) PREPARATION FOR RADIO PROTECTION

(71) Applicant: LYCORED LTD., Beer-Sheva (IL)

(72) Inventors: Vladimir A. TYURENKOV, Moscow (RU); Alexey A. TYURENKOV, Moscow (RU); Yuriy D. TURIYANSKY, Zaporizhzhya (UA)

(21) Appl. No.: 14/653,543

(22) PCT Filed: Dec. 19, 2012
(86) PCT No.: PCT/UA2012/000114

§ 371 Date: Jun. 18, 2015

(51) Int. Cl. A61K 36/06

(52) U.S. Cl. CPC A61K 36/06

(57) ABSTRACT

The invention is related to the field of medicine, particularly to pharmacology, and disclose the usage of carotenoid phytoene in the composition of lipid complex which is extracted from the fungal biomass of Blakeslea trispora as a preparation for the radio protection.

The invention is related to the field of medicine, particularly to pharmacology, and can be used in pharmaceutical industry for manufacturing of medical and preventive preparations of a radio protective effect.

It is known a substance which has a radio protective effect [1], which contains a lipophilic complex of black chokeberry.

The imperfection of this invention is the lack of knowledge on the active substance (or substances) of the lipophilic complex of the black chokeberry, which influences on its radio protective characteristics. Considering the variability of the characteristics of the black chokeberry can be rather essential under the different weather conditions of planting, the efficiency of the usage of the lipophilic complex may be quite different, and that can bring to impossibility of guaranteeing a stable positive effect for such substance.

As a prototype a patent [2] has been considered, according to which Saint-Mary-thistle Silybum marianum is used as a radio protective preparation.

The similar drawbacks as for the previous invention are relevant to this one, videlicet: impossibility to determine exactly the substance (or a complex of substances) with a clear chemical formula and effective dosage which securely facilitates the radio protection.

The aim of retrieving of a radio protective preparation was the identification of an ingredient in the structure of a natural substance which has highly treating and biological activity.

The above aim is resolving by using carotenoid phytoene in the composition of lipid complex, which is extracted from the fungal biomass of Blakeslea trispora, as a preparation for the radio protection.

The usage of carotenoid phytoene in the composition of the lipid complex, which is extracted from the biomass of the phytoene synthesizing strain of the mycelium fungus Blakeslea trispora [3, 4], as the radio protective preparation, guarantees natural plant origin of that compound and assures for such radio protective preparation high attractiveness and biological value, as also ensures the presence of the lipids in the extract which are synthesized by the fungus and appeared to be the natural medium which carotenoid phytoene is synthesized in.

The efficiency of the usage of the lipid complex with phytoene which is obtained from the biomass of the Blakeslea trispora fungus and investigation of its radio protective abilities has been determined in the trials with biological membrane models.

The biologic membranes play the most important role in cell vital activity. The high sensibility of lipids and phospholipids, which are the main structural components of biomembranes, as to oxidative stress, including those caused by irradiation, is the consequence of the high content of unsaturated fatty acids. The liposomes being obtained from phospholipids of hen's egg yolk were used as a model of the biomembranes. The phospholipids are able to form liposome emulsions in water; this allows studying in model experiments the radioprotective and antioxidant properties of various water-soluble and water-insoluble compounds, including water-insoluble phytoene as well.

To obtain the liposomes, 20 mg of the phospholipids, being dissolved in chloroform, were entered into a conical vial and dried in vacuum. Then 2 ml of a saline solution containing 0.15 M NaCl, 0.005 M KCl, 0.0012 M KH₂PO₄, 0.0012 M Na₂HPO₄ at pH 7.4 was added to the dried sample. The mixture has been shaken, input into an ultrasonic bath for 30 seconds, and then the total volume was increased up to 10 ml, stirred, treated with ultrasound, and as the result the liposome suspension with the phospholipid concentration of 2 mg/ml was received.

To obtain the liposomes with various concentrations of phytoene in their structures, the solutions of phospholipids in chloroform and phytoene in the composition of the lipid complex of the Blakeslea trispora fungal biomass were mixed, this mixture was dried, suspended in the saline solution with the further ultrasonic treatment, to obtain the suspension of the liposomes with phytoene in the ratio of phytoene : phospholidid as 1:10 or 1:100.

The influence of the ionizing radiation on the water molecules in aerating conditions induces active OH* radical, which is an active oxidant and is able to induce processes of phospholipid peroxidation in the liposomes. To conduct the radiation induced oxidation of the liposomes, 1 ml of the liposomal emulsion containing 2 mg/ml of the phospholipids and various concentrations of phytoene was irradiated with the dose of 500 Gy by Co⁶° γ-quanta, under the dosage rate of 84.8 Gy/min in the saline solution.

Due to OH* radical arising as the result of a decay of the organic peroxides in the presence of Fe⁺⁺ ions, the reactions of the phospholipid peroxidation is induced in the liposomes as well. The liposome peroxidation was initiated by adding of 0.05 ml of 1.0 mM FeSO₄ solution, which made the final concentration of Fe⁺⁺ equals to 50 μM. The mixture was accurately stirred and incubated for 30 minutes under the room temperature.

The efficacy of the radiative and Fe⁺⁺-induced oxidation of the liposome emulsion was estimated by malondialdehyde (MDA) outcome, which is one of the final products of the lipid peroxidation. After finishing of the irradiation or the induced oxidation reaction, 2 ml of the combined solution which consists of 15% solution of trichloroacetic acid, 0.375% solution of thiobarbituric acid (TBA), 0.25 M HCl as also of 0.005% EDTA and 0.00025% ionole were added to 1 ml of the liposome emulsion. The obtained mixture was heated in a boiling water bath for 20 minutes, then cooled down and purified by centrifugation for 15 minutes at 2000 rpm. Optical density of the colored TBA products was calculated by the optical density data, considering the molar extinction coefficient at 532 nm equals to 156,000 mol⁻¹·cm⁻¹.

The presence of chemical agents in the suspended medium may decrease MDA formation induced by the irradiation or Fe⁺⁺; this proves the presence of radio protective or antioxidant properties in these compounds. Taking as 100% the level of the MDA increment at the induced liposome oxidation above spontaneous phospholipids oxidation in the absence of phytoene, and taking into account the fact that the presence of phytoene in the liposomes can modify the level of the spontaneous oxidation, the protection efficacy (PE)_{OK} in the percence of the induced liposome oxidation was calculated by the formula:

(PE)_{OK}=(CP_{OK}−CP_{K})·100/(C_{OK}−C_{K}),

in which CP_{K} , CP_{OK} are the MDA concentrations in the liposomes with phytoene before and after the liposome induced oxidation respectively; C_{K}, C_{OK} (are the MDA concentrations in the control liposomes (without phytoene) before and after the liposome induced oxidation respectively.

The results are shown in the Table.

TABLE
Anti irradiation and anti oxidation properties of phytoene

| | | Protection against Fe⁺⁺- |
|---|---|---|
| Phytoene:phospholipid | Radioprotection in % | induced oxidation in % |
| 1:100 | 37.2 ± 5.2 | 18.7 ± 11.5 |
| 1:10 | 62.0 ± 10.4 | 50.9 ± 6.6 |

The performed experiments demonstrated that phytoene protects the liposomes against the oxidative stress being induced by the radiation or with the ferrous iron ions.

The advantage of the proposed preparation are in containing of the active substance which is of the determined composition and structure and allows to dose efficiently the radio protective preparation and therefore actively facilitate for the protection against the radioactive irradiation. The chosen source of the raw material consists of the natural ingredients which has a big meaning for the treatment-and-preventive and therapeutic influence of the preparation.

The radio protective preparation is obtained as follows.

The dried biomass of the Blakeslea trispora fungus is obtained from the phytoene strain as for example according to the patent [3] by the fermentation technology as it is shown e.g. in the patent [4]. Then the biomass is extracted in the oil for example under the technology being described in the patent [5]. The obtained oil-lipid complex with phytoene is introduced into a composition of medical preparations which are in adipose forms, and after that processing into the final formulation like e.g. liquid solutions, soft-gel capsules etc is undertaken.

### REFERENCES

1. Patent of Ukraine No. 19231, A61K 36/73, A61P 17/16, published 25 Dec. 1997, Bulletin No 6.
2. Patent of Ukraine No. 29568, A61K 35/78, published 15 Nov. 2000, Bulletin No 6.
3. Patent of Ukraine No. 85489, C12N 1/11, C12P 23/00, C12N 15/01, C12R 1/645, published 11 Mar. 2008, Bulletin No 5.
4. Patent of Ukraine No. 86556, C12P 23/00, C12N 1/14, published 27 Apr. 2009, Bulletin No 8.
5. Patent of Ukraine No. 82090, C07C 403/00, A23L 1/302, A61K 31/07, C09B 61/00, C12P 23/00, published 15 Feb. 2006, Bulletin No 2.

1. The usage of carotenoid phytoene in the composition of lipid complex which is extracted from the fungal biomass of Blakeslea trispora as a preparation for the radio protection.
